(19) United States
(12) Patent Application Publication
(10) Pub. No.: US 2026/0022105 A1
(43) Pub. Date: Jan. 22, 2026

(54) Method for Preparing 2,5-Furandicarboxylic Acid

(71) Applicant: NATIONAL SUN YAT-SEN UNIVERSITY, Kaohsiung City (TW)

(72) Inventors: Ken-Lin Chang, Kaohsiung City (TW); Quang-Tam Huynh, Kaohsiung City (TW); Bo-Yi Chen, Kaohsiung City (TW)

(21) Appl. No.: 18/925,075

(22) Filed: Oct. 24, 2024

(30) Foreign Application Priority Data
Jul. 18, 2024 (TW) 113126962

(51) Int. Cl. C07D 307/68; B01J 21/12; B01J 23/889

(52) U.S. Cl. CPC C07D 307/68; B01J 21/12; B01J 23/8892

(57) ABSTRACT

A method for preparing 2,5-furandicarboxylic acid includes the steps of mixing a biomass material, a dehydration catalyst and a first solvent to obtain a dehydration precursor; conducting a dehydration reaction on the dehydration precursor at 120-170° C. for 30-250 minutes to obtain a dehydration mixture containing 5-hydroxymethylfurfural; mixing the dehydration mixture, an oxidation catalyst, an oxidant and a second solvent to obtain an oxidation precursor; and conducting an oxidation reaction on the oxidation precursor at 60-90° C. for 2-12 hours to obtain an oxidation mixture containing 2,5-furandicarboxylic acid. The biomass material contains cellulose. The first solvent contains an ionic liquid, dimethyl sulfoxide and deionized water. The oxidation catalyst is a solid copper-manganese nanomaterial.

### CROSS REFERENCE TO RELATED APPLICATION

The application claims the benefit of Taiwan Application Serial No. 113126962, filed on Jul. 18, 2024, and the entire contents of which are incorporated herein by reference.

### BACKGROUND OF THE INVENTION

### 1. Field of the Invention

The present invention relates to a method for preparing 2,5-furandicarboxylic acid and, more particularly, to a method for preparing 2,5-furandicarboxylic acid using biomass material as a raw material.

### 2. Description of the Related Art

2,5-furandicarboxylic acid (abbreviated as FDCA, with chemical structure shown in FIG. 1) is a biodegradable material. The U.S. Department of Energy has listed FDCA as one of the 12 preferred chemicals for the future construction of a green chemical industry. Compared to polyethylene terephthalate (PET), polyethylene 2,5-furandicarboxylic acid (PEF) formed by polymerization of FDCA and ethylene glycol exhibits better thermal stability, gas barrier property and mechanical strength. Moreover, the energy consumption and carbon emissions of the process for preparing PEF with FDCA are lower than those for preparing PET. Thus, PEF can not only replace the petroleum-based plastic products but also mitigate the environmental impact of plastic products. Therefore, FDCA has a broad application prospect in the field of green chemistry.

In the known method for preparing FDCA, 5-hydroxymethylfurfural (abbreviated as 5-HMF) is used as a starting material and reacts with noble metal catalysts containing gold, palladium or platinum under a strong alkaline or high temperature environment. However, the reaction conditions of the above preparing method are harsh, and both 5-HMF and the noble metal catalysts are expensive chemicals, resulting in high production cost of FDCA. Moreover, the noble metal catalysts often suffer from stability issues, as they tend to leach into the reaction mixture, which require additional extraction to recover the noble metal catalysts, thus making the preparing process of FDCA complicated.

In view of this, it is necessary to improve the known method for preparing FDCA.

### SUMMARY OF THE INVENTION

To solve the above problems, it is an objective of the present invention to provide a method for preparing 2,5-furandicarboxylic acid, which can reduce the production cost of 2,5-furandicarboxylic acid.

It is another objective of the present invention to provide method for preparing 2,5-furandicarboxylic acid by using base metal catalysts with strong bond strength, which eliminates the need for additional extraction steps to recover the catalysts.

It is yet another objective of the present invention to provide method for preparing 2,5-furandicarboxylic acid, which can achieve a high yield of 2,5-furandicarboxylic acid with base metal catalysts and raw biomass materials.

As used herein, the term “a”, “an” or “one” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

A method for preparing 2,5-furandicarboxylic acid according to the present invention includes the steps of mixing a biomass material, a dehydration catalyst and a first solvent to obtain a dehydration precursor; conducting a dehydration reaction on the dehydration precursor at 120-170° C. for 30-250 minutes to obtain a dehydration mixture containing 5-hydroxymethylfurfural; mixing the dehydration mixture, an oxidation catalyst, an oxidant and a second solvent to obtain an oxidation precursor; and conducting an oxidation reaction on the oxidation precursor at 60-90° C. for 2-12 hours to obtain an oxidation mixture containing 2,5-furandicarboxylic acid. The biomass material contains cellulose. The first solvent contains an ionic liquid, dimethyl sulfoxide and deionized water. The oxidation catalyst is a solid copper-manganese nanomaterial.

Therefore, the method for preparing 2,5-furandicarboxylic acid of the present invention allows the use of biomass material containing cellulose or lignocellulose as starting materials. Without the need for chemical pretreatment, high-value 2,5-furandicarboxylic acid (FDCA) can be formed through a two-step one-pot reaction. This method not only eliminates the need for the purification step of 5-hydroxymethylfurfural (5-HMF) and the use of noble metal catalysts, but also enables the synthesis of FDCA under mild conditions without strong bases or high pressure. Additionally, since the oxidation catalyst is a solid catalyst, it can be separated from the FDCA dissolved in the second solvent by simple filtration. The separated oxidation catalyst can be recycled and reused in catalyzing the oxidation reaction. Furthermore, the one-pot reaction system can simplify the complicated process for producing FDCA, thereby reducing its production cost.

In an example, the biomass material is jackfruit peel, pineapple stem, rice stalk or bagasse. Thus, since the biomass material is derived from agricultural waste, utilizing the biomass material as starting materials to synthesize FDCA can provide new economic value to the agricultural waste, thereby enhancing the added value of the agricultural waste.

In an example, the dehydration catalyst is a solid acid catalyst. Thus, the dehydration catalyst is insoluble in the liquid 5-HMF, allowing for the easy separation of the dehydration catalyst from 5-HMF, and the separated dehydration catalyst can be recycled and reused in reaction, thereby reducing the yield loss of 5-HMF and reducing the production cost.

In an example, after obtaining the dehydration mixture, the dehydration catalyst is separated from the dehydration mixture by filtration. Thus, filter paper or filter mesh with different pore sizes can be used for separation, thereby simplifying the process for producing 5-HMF.

In an example, by weight percentage, the dehydration precursor contains 0.13-3.68% of the biomass material, 0.78-4.50% of the dehydration catalyst and 92.10-99.08% of the first solvent. It was confirmed through experimental results that with the above amounts of components, a higher yield of 5-HMF can be achieved.

In an example, by volume percentage, the first solvent contains 0.8-4.6% of the ionic liquid, 24.4-74.1% of dimethyl sulfoxide and 24.4-74.1% of deionized water. It was confirmed through experimental results that with the above amounts of components, a higher yield of 5-HMF can be achieved.

In an example, the ionic liquid is 1-allyl-3-methylimidazolium chloride ([AMIM]Cl), 1-butyl-3-methylimidazolium chloride ([BMIM]Cl), 1-butyl-3-methylimidazolium hydrogen sulfate ([BMIM]HSO₄) or 1-butyl-3-methylimidazolium tetrafluoroborate ([BMIM]BF₄). Thus, by using environmentally friendly ionic liquids, environmental pollution can be reduced.

In an example, the ionic liquid is 1-butyl-3-methylimidazolium hydrogen sulfate ([BMIM]HSO₄). Thus, the hydrogen sulfate anion dissociated from the ionic liquid can provide an acidic reaction environment for the cellulose to hydrate and form 5-HMF more readily, thereby enhancing the yield of 5-HMF.

In an example, after obtaining the oxidation mixture, the oxidation catalyst is separated from the oxidation mixture by filtration. Thus, filter paper or filter mesh with different pore sizes can be used to separate the oxidation catalyst, thereby simplifying the process for producing FDCA.

In an example, by weight percentage, the oxidation precursor contains 25.66-69.51% of the dehydration mixture, 0.56-4.17% of the oxidation catalyst, 5.80-31.67% of the oxidant and 18.64-59.99% of the second solvent. It was confirmed through experimental results that with the above amounts of components, a higher yield of FDCA can be achieved.

In an example, the oxidant is oxygen, hydrogen peroxide, potassium permanganate, sodium chloride or tert-butyl hydroperoxide. Thus, by selecting a low-cost and commonly used oxidant for the oxidation reaction, the production cost of FDCA can be reduced.

In an example, the oxidant is tert-butyl hydroperoxide. Thus, the oxidant can gently oxidize 5-HMF in the dehydration mixture into FDCA, preventing excessive oxidation of 5-HMF into undesired by-products, thereby increasing the yield of FDCA.

In an example, the second solvent is water, acetonitrile, ethyl acetate or tert-butanol. Thus, by selecting a low-cost and commonly used solvent as the second solvent, the production cost of FDCA can be reduced.

In an example, the second solvent is acetonitrile. Thus, it can not only enhance the interaction of 5-HMF and the oxidation catalyst, but also prevent the oxidation catalyst from losing its catalytic activity, thereby increasing the yield of FDCA.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a chemical structure of 2,5-furandicarboxylic acid.

FIG. 2 is a flow chart of a method for preparing 2,5-furandicarboxylic acid according to an embodiment of the present invention.

FIG. 3 is a bar chart illustrating the yield of 5-HMF in groups A1 to A4 in Experiment (A).

FIG. 4 is a bar chart illustrating the yield of 5-HMF in groups B1-1 to B1-6 and B2-1 to B2-6 in Experiment (B).

FIG. 5 is a bar chart illustrating the yield of 5-HMF in groups C1 to C6 in Experiment (C).

FIG. 6 is a bar chart illustrating the yield of 5-HMF in groups D1 to D5 in Experiment (D).

FIG. 7 is a bar chart illustrating the conversion rate of 5-HMF and the yield of FDCA in groups E1 to E4 in Experiment (E).

FIG. 8 is a bar chart illustrating the conversion rate of 5-HMF and the yield of FDCA in groups F1 to F5 in Experiment (F).

FIG. 9 is a bar chart illustrating the conversion rate of 5-HMF and the yield of FDCA in groups G1 to G4 in Experiment (G).

FIG. 10 is a bar chart illustrating the conversion rate of 5-HMF and the yield of FDCA in groups H1 to H4 in Experiment (H).

FIG. 11 is a line chart illustrating the conversion rate of 5-HMF and the yield of FDCA in groups I1 to I4 in Experiment (I).

FIG. 12 is a line chart illustrating the conversion rate of 5-HMF and the yield of FDCA in groups J1 to J6 in Experiment (J).

FIG. 13 is a bar chart illustrating the conversion rate of 5-HMF and the yield of FDCA in groups K1 to K4 in Experiment (K).

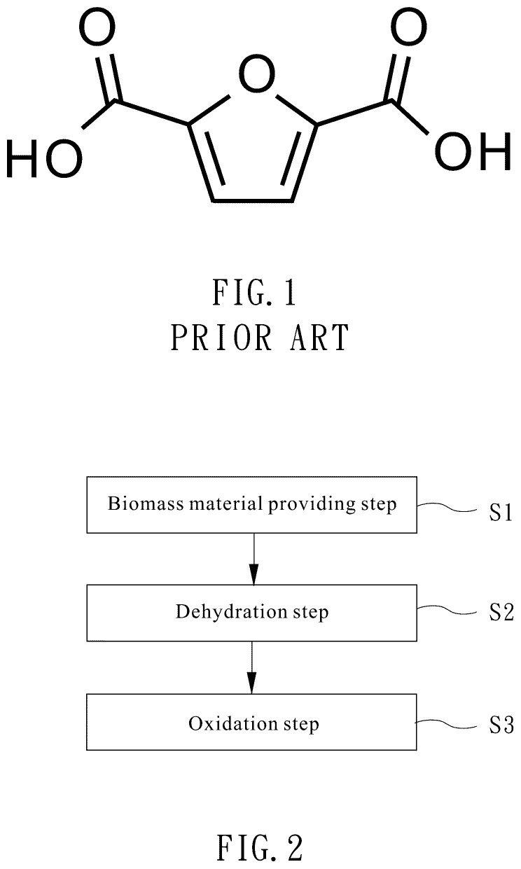

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

### DETAILED DESCRIPTION OF THE INVENTION

In order to make the above and other objectives, features, and advantages of the present invention clearer and easier to understand, the preferred embodiments of the present invention will be described hereinafter in connection with the accompanying drawings. Furthermore, the elements designated by the same reference numeral in various figures will be deemed as identical, and the description there of will be omitted.

With reference to FIG. 2, the method for preparing 2,5-furandicarboxylic acid of the present invention can include a biomass material providing step S1, a dehydration step S2, and an oxidation step S3, thereby enabling a one-pot method to first convert a biomass material into 5-HMF through a dehydration reaction and then convert the 5-HMF into FDCA through an oxidation reaction.

In the biomass material providing step S1, the biomass material is provided. The biomass material can contain cellulose. For example, the present invention can use any biomass material containing cellulose or lignocellulose. More specifically, the biomass material can be peels of jackfruit (Artocarpus heterophyllus), stems of pineapple (Ananas comosus), stalks of rice (Oryza sativa) or bagasse (i.e., the residue left after sugarcane (Saccharum) is crushed and juiced). Preferably, the biomass material is washed, dried and grounded to prevent large particle size that may reduce the efficiency of subsequent dehydration and oxidation reaction. In this embodiment, the washed biomass material can be dried at 60° C. for 24 hours, and after grinding, the biomass material is ensured to pass through a 40-mesh sieve so that the particle size can be less than 0.42 mm.

In the dehydration step S2, the biomass material, a dehydration catalyst and a first solvent can be mixed to obtain a dehydration precursor. The dehydration catalyst is used to catalyze the dehydration reaction of the biomass material. The first solvent is used to dissolve the cellulose in the biomass material and to uniformly disperse the dehydration catalyst in the first solvent. The mixing ratio of each component in the dehydration precursor can be adjusted according to needs. In this embodiment, by weight percentage, the dehydration precursor contains 0.13-3.68% of the biomass material, 0.78-4.50% of the dehydration catalyst and 92.10-99.08% of the first solvent.

Specifically, the dehydration catalyst can be a solid acid catalyst, preferably a silica-alumina composite (SiO₂/Al₂O₃). In this embodiment, the silica-alumina composite is obtained by mixing sodium metasilicate (Na₂SiO₃), aluminum trichloride (AlCl₃), sodium hydroxide (NaOH), hydrochloric acid (HCl) and deionized water (DI water), followed by stirring, crystallization and calcination. Due to Lewis acid character of the silica-alumina composite, the silica-alumina composite can facilitate depolymerization of the biomass material, thereby enhancing the yield of 5-HMF in the dehydration reaction.

The first solvent can contain an ionic liquid, dimethyl sulfoxide (DMSO) and deionized water. The ionic liquid can facilitate the conversion of the biomass material into 5-HMF by disrupting intramolecular and intermolecular hydrogen bonds of cellulose. DMSO can prevent 5-HMF from nucleophilic attack due to its strong affinity for 5-HMF, thereby inhibiting the formation of by-products (e.g., humins) and thus enhancing the yield of 5-HMF in the dehydration reaction. For example, the ionic liquid can be 1-allyl-3-methylimidazolium chloride ([AMIM]Cl), 1-butyl-3-methylimidazolium chloride ([BMIM]Cl), 1-butyl-3-methylimidazolium hydrogen sulfate ([BMIM]HSO₄) or 1-butyl-3-methylimidazolium tetrafluoroborate ([BMIM]BF₄). Preferably, the ionic liquid can be [BMIM]HSO₄. The hydrogen sulfate anion (HSO₄⁻) dissociated from [BMIM]HSO₄ provides an acidic reaction environment for the cellulose of the biomass material to hydrate and form 5-HMF more readily. In this embodiment, by volume percentage, the first solvent contains 0.8-4.62% of [BMIM]HSO₄, 24.4-74.1% of DMSO and 24.4-74.1% of deionized water.

Next, the dehydration precursor can be stirred at 120-170° C. to conduct the dehydration reaction for 30-250 minutes, thereby obtaining a dehydration mixture containing 5-HMF.

Notably, the dehydration catalyst can be separated from the dehydration mixture before the subsequent oxidation reaction of the dehydration mixture. Moreover, since the dehydration catalyst is a solid, the dehydration catalyst can be easily separated by filtration. For example, filter paper or filter mesh with different pore sizes can be selected according to the particle size of the dehydration catalyst for separation. Thus, the yield loss of 5-HMF during the separation process can be reduced, and furthermore, the separated dehydration catalyst can be recycled and reused in the preparation of the dehydration mixture containing 5-HMF.

In the oxidation step S3, the dehydration mixture obtained in the dehydration step S2 can be directly mixed with an oxidation catalyst, an oxidant and a second solvent to obtain an oxidation precursor. The oxidation catalyst is used to catalyze the oxidation reaction of the dehydration mixture. The oxidant is used to oxidize the 5-HMF in the dehydration mixture. The second solvent is used to uniformly mix the dehydration mixture, the oxidation catalyst and the oxidant in the second solvent to form the oxidation precursor. The mixing ratio of each component in the oxidation precursor can be adjusted according to needs. In this embodiment, by weight percentage, the oxidation precursor contains 25.66-69.51% of the dehydration mixture, 0.56-4.17% of the oxidation catalyst, 5.80-31.67% of the oxidant and 18.64-59.99% of the second solvent.

Specifically, the oxidation catalyst can be a solid copper-manganese nanomaterial. In this embodiment, Cu—Mn₂O₄ is used as the oxidation catalyst. Cu—Mn₂O₄ can be obtained by mixing copper (II) nitrate (Cu(NO₃)₂), manganese (II) sulfate (MnSO₄), citric acid, polyethylene glycol (PEG) and methanol, followed by stirring and calcination. Thus, by using a solid copper-manganese nanomaterial as the oxidation catalyst, the use of noble metal catalysts can be avoided, and furthermore, the oxidation catalyst can be easily separated by filtration. For example, filter paper or filter mesh with different pore sizes can be selected according to the particle size of the oxidation catalyst for separation. The separated oxidation catalyst can be recycled and reused in the preparation of FDCA, thereby simplifying the preparing process of FDCA and reducing the production cost of FDCA.

The oxidant refers to a substance that gains electrons in an oxidation-reduction reaction, i.e., a substance whose oxidation state decreases from a higher state to a lower state, such as oxides, peroxides, halogens, etc.; it is understood by a person skilled in the art, and the description thereof will be omitted. Preferably, the oxidant can be a mild oxidant, such as tert-butyl hydroperoxide (tBuOOH), hydrogen peroxide (H₂O₂), etc. Thus, by using a mild oxidant for the oxidation reaction, excessive oxidation of 5-HMF in the dehydration mixture can be avoided, preventing its conversion into undesired by-products and thereby increasing the yield of FDCA.

The second solvent can be water or an organic solvent, such as ethyl acetate (EtOAc), tert-butanol (tBuOH), acetonitrile (MeCN), etc. In this embodiment, acetonitrile is used as the second solvent. The low viscosity of acetonitrile can facilitate the attachment of 5-HMF in the dehydration mixture to the active site of the oxidation catalyst. Moreover, the polarity of acetonitrile is favorable for dissolving both organic matter (i.e., the dehydration mixture) and inorganic matter (i.e., the oxidation catalyst). Thus, the interaction between 5-HMF in the dehydration mixture and the oxidation catalyst can be facilitated, thereby enhancing the yield of FDCA. Additionally, acetonitrile can act as Lewis base to stabilize Cu⁺ ions of the oxidation catalyst, preventing the Cu⁺ ions from disproportionation to form Cu²⁺ ions and metallic copper (which would cause the oxidation catalyst to lose catalytic activity), thereby stabilizing the oxidation catalyst.

Next, the oxidation precursor can be stirred at 60-90° C. to conduct the oxidation reaction for 2-12 hours, thereby obtaining an oxidation mixture containing FDCA.

In order to prove that the method for preparing 2,5-furandicarboxylic acid of the present invention can effectively convert biomass material into FDCA, the following experiments were performed.

### (A) Effect of Different Ionic Liquids on the Yield of 5-HMF

In this experiment, four different ionic liquids were respectively added to a mixture of 0.2 g of jackfruit peel, 0.121 g of SiO₂/Al₂O₃, 7.5 mL of DMSO and 2.5 mL of deionized water, and then the dehydration reaction was conducted at 150° C. for 120 minutes.

Next, the concentration of 5-HMF in the obtained dehydration mixture was analyzed by high performance liquid chromatography (HPLC) equipped with UV-Vis detector. The yield of 5-HMF in each group was calculated according to the following equation (1):

$$Yield ( \% ) = \frac{{C}_{HMF} \times V \times 162.14 \times 1 {0}^{- 6}}{126.11 \times M \times A \div 100} \times 100 \% \qquad ( Equation 1 )$$

In the above equation (1), “C_{HMF}” represents the concentration of 5-HMF obtained from HPLC analysis (unit: mg/mL); “V” represents the volume of the dehydration mixture obtained at the end of the dehydration reaction (unit: mL); “162.14” is the molecular weight of cellulose; “126.11” is the molecular weight of 5-HMF; “M” is the mass of the biomass material (unit: mg); and “A” is the percentage of cellulose in the biomass material.

It should be noted that “A (percentage of cellulose)” in the equation (1) was obtained by following steps: mixing 300 mg of the biomass material with 3 mL of 72% sulfuric acid and reacting at 30° C. for 60 minutes to obtain a hydrolysate; mixing the hydrolysate with 84 mL of deionized water to obtain a diluted product, and then sterilizing the diluted product in an autoclave at 121° C.; vacuum filtering the sterilized diluted product to obtain a filtrate; neutralizing the filtrate with calcium carbonate to adjust the pH to between 5 and 6; and filtering out the calcium carbonate from the filtrate, and then analyzing the percentage of cellulose in the filtrate by HPLC. The percentage of cellulose in the biomass material was calculated according to the following equation (2):

$$Percentage of Cellulose ( \% ) = \frac{{C}_{cellulose} \times 0.087}{300} \times 100 \% \qquad ( Equation 2 )$$

In the above equation (2), “C_{celluose}” represents the concentration of cellulose obtained from HPLC analysis (unit: mg/L); “0.087” is the total volume of the filtrate (unit: L); “M” is the mass of the cellulose (unit: mg).

TABLE 1
Experimental results of different ionic liquids

| | | Ionic liquid amount | Yield of 5-HMF |
|---|---|---|---|
| Group | Ionic liquid | (mL) | (%) |
| A1 | [BMIM]HSO₄ | 0.121 | 73.86 |
| A2 | [BMIM]BF₄ | 0.121 | 2.69 |
| A3 | [BMIM]Cl | 0.121 | 54.87 |
| A4 | [AMIM]Cl | 0.121 | 6.17 |

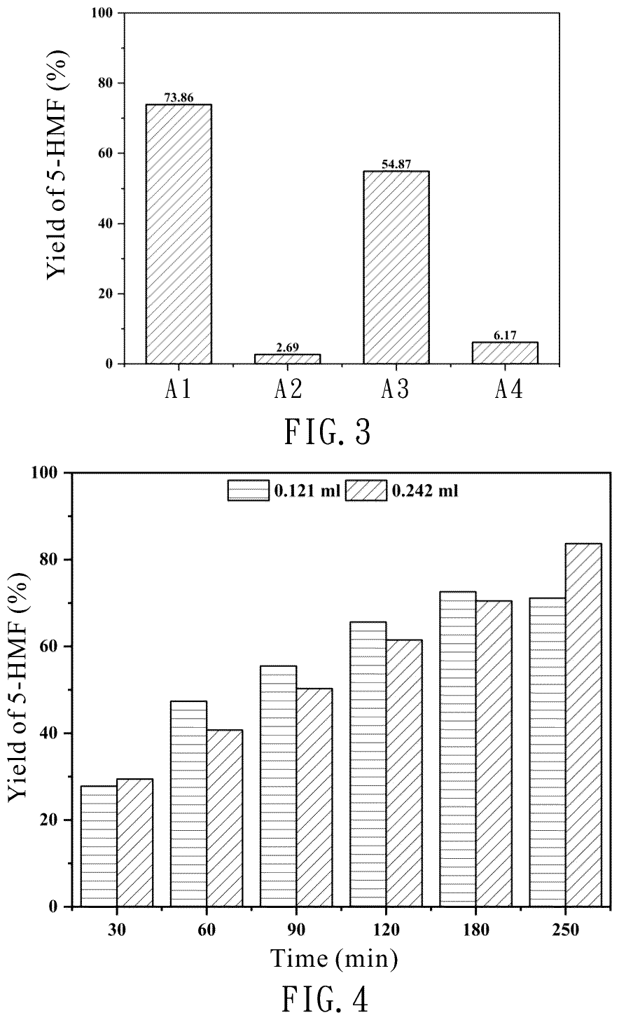

Referring to Table 1 and FIG. 3, among groups A1 to A4, group A1, which used [BMIM]HSO₄ as the ionic liquid, exhibited the highest yield of 5-HMF.

### (B) Effect of Ionic Liquid Amount on the Yield of 5-HMF

In this experiment, 0.121 mL or 0.242 mL of [BMIM]HSO₄ was respectively added to a mixture of 0.2 g of jackfruit peel, 0.121 g of SiO₂/Al₂O₃, 7.5 mL of DMSO and 2.5 mL of deionized water, and then the dehydration reaction was conducted at 150° C. for 30-250 minutes.

Next, the concentration of 5-HMF in the obtained dehydration mixture was analyzed by HPLC equipped with UV-Vis detector. The yield of 5-HMF in each group was calculated according to the above equation (1).

TABLE 2
Experimental results of different amounts of ionic liquids

| | Ionic liquid amount | Time | Yield of 5-HMF |
|---|---|---|---|
| Group | (mL) | (min) | (%) |
| B1-1 | 0.121 | 30 | 27.79 |
| B1-2 | | 60 | 47.38 |
| B1-3 | | 90 | 55.49 |
| B1-4 | | 120 | 65.61 |
| B1-5 | | 180 | 72.59 |
| B1-6 | | 250 | 71.13 |
| B2-1 | 0.242 | 30 | 29.44 |
| B2-2 | | 60 | 40.71 |
| B2-3 | | 90 | 50.29 |
| B2-4 | | 120 | 61.48 |
| B2-5 | | 180 | 70.48 |
| B2-6 | | 250 | 83.68 |

Referring to Table 2 and FIG. 4, among the different ionic liquid amounts and reaction times, group B2-6, which used 0.242 mL of [BMIM]HSO₄ and reacted for 250 minutes, exhibited the highest yield of 5-HMF.

### (C) Effect of Reaction Temperature on the Yield of 5-HMF

In this experiment, a mixture of 0.2 g of jackfruit peel, 0.121 g of SiO₂/Al₂O₃, 7.5 mL of DMSO, 0.242 mL of [BMIM]HSO₄ and 2.5 mL of deionized water was used to conduct the dehydration reaction at 120-170° C. for 180 minutes.

Next, the concentration of 5-HMF in the obtained dehydration mixture was analyzed by HPLC equipped with UV-Vis detector. The yield of 5-HMF in each group was calculated according to the above equation (1).

TABLE 3
Experimental results of dehydration
reactions at different temperatures

| Group | Temperature (° C.) | Yield of 5-HMF (%) |
|---|---|---|
| C1 | 120 | 47.17 |
| C2 | 130 | 60.56 |
| C3 | 140 | 71.21 |
| C4 | 150 | 73.86 |
| C5 | 160 | 68.51 |
| C6 | 170 | 55.17 |

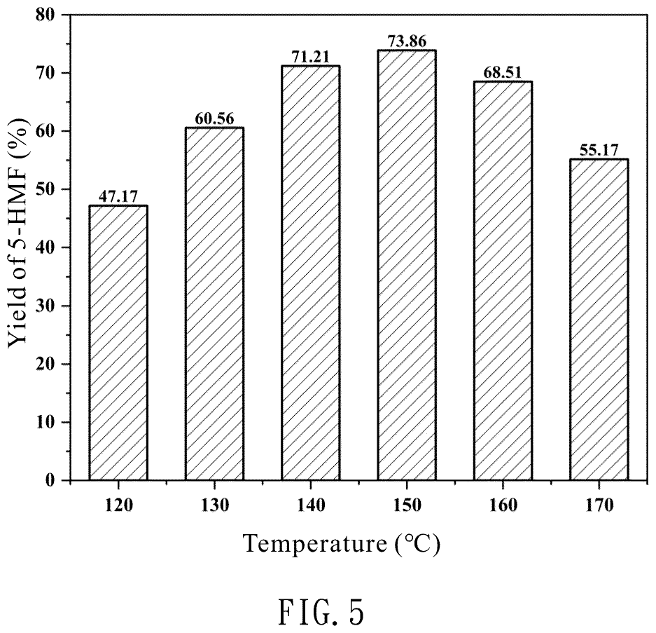

Referring to Table 3 and FIG. 5, among groups C1 to C6, group C4, which reacted at 150° C., exhibited the highest yield of 5-HMF.

### (D) Effect of Reaction Time on the Yield of 5-HMF

In this experiment, a mixture of 0.2 g of jackfruit peel, 0.121 g of SiO₂/Al₂O₃, 7.5 mL of DMSO, 0.242 mL of [BMIM]HSO₄ and 2.5 mL of deionized water was used to conduct the dehydration reaction at 150° C. for 60-300 minutes.

Next, the concentration of 5-HMF in the obtained dehydration mixture was analyzed by HPLC equipped with UV-Vis detector. The yield of 5-HMF in each group was calculated according to the above equation (1).

TABLE 4
Experimental results of different reaction times

| Group | Time (min) | Yield of 5-HMF (%) |
|---|---|---|
| D1 | 60 | 48.41 |
| D2 | 120 | 64.73 |
| D3 | 180 | 72.69 |
| D4 | 240 | 72.30 |
| D5 | 300 | 63.55 |

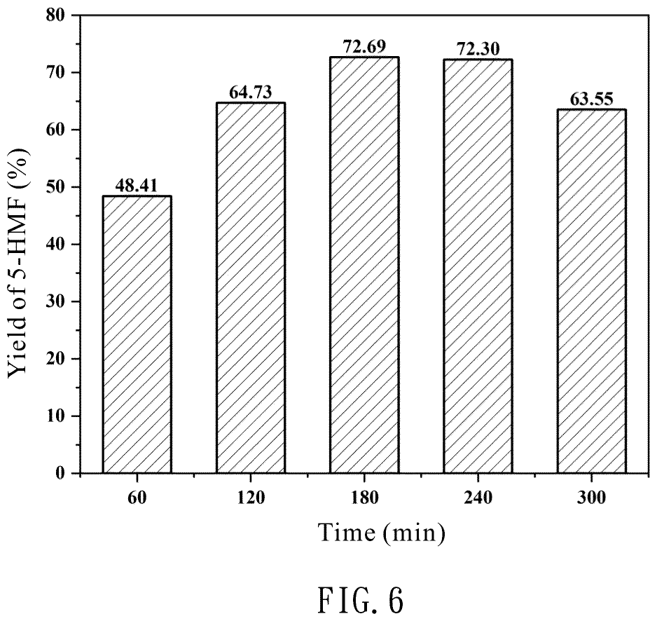

Referring to Table 4 and FIG. 6, among groups D1 to D6, group D3, which reacted for 180 minutes, exhibited the highest yield of 5-HMF.

### (E) Effect of Second Solvent on the Yield of FDCA

In this experiment, different second solvents were respectively added to a mixture of 2.5 mL of the dehydration mixture, 0.5 mL of tert-butyl hydroperoxide and 50 mg of Cu—Mn₂O₄, and then the oxidation reaction was conducted at 70° C. for 12 hours.

Next, the moles of 5-HMF and FDCA in the obtained oxidation mixture were analyzed by HPLC equipped with UV-Vis detector. The conversion rate of 5-HMF (%) and the yield of FDCA (%) in each group were calculated according to the following equations (3) and (4), respectively.

$$( Equation 3 )$$

$$Conversion rate of 5 - HMF ( \% ) = ( 1 - \frac{Moles of 5 - HMF after reaction}{Moles of 5 - HMF before reaction} ) \times 100 \%$$

$$( Equation 4 )$$

$$Yield of FDCA ( \% ) = \frac{Moles of FDCA}{Moles of 5 - HMF before reaction} \times 100 \%$$

TABLE 5
Experimental results of different second solvents

| | | Second | | |
|---|---|---|---|---|
| | | solvent | Conversion | Yield of |
| | Second | amount | rate of 5-HMF | FDCA |
| Group | solvent | (mL) | (%) | (%) |
| E1 | Water | 2.5 | 100 | 34.07 |
| E2 | Acetonitrile | 2.5 | 100 | 78.64 |
| | (MeCN) | | | |
| E3 | Ethyl acetate | 2.5 | 100 | 45.77 |
| E4 | Tert-butanol | 2.5 | 100 | 57.29 |
| | (TBA) | | | |

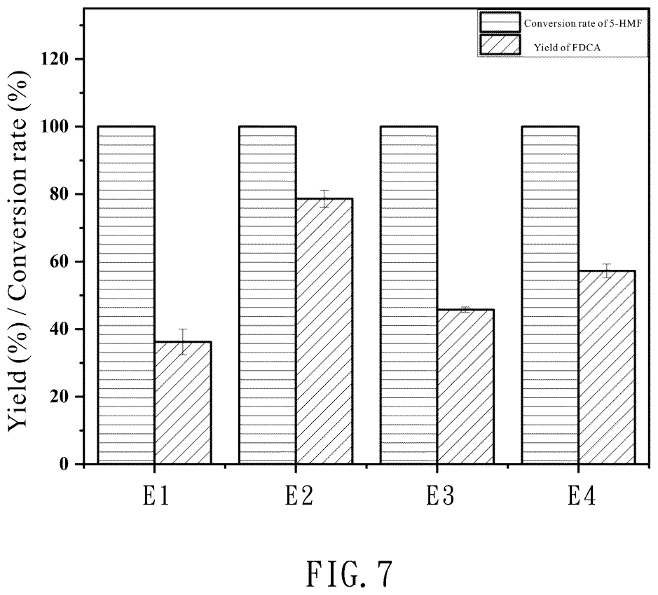

Referring to Table 5 and FIG. 7, among groups E1 to E4, group E2, which used acetonitrile as the second solvent, exhibited the highest yield of FDCA.

### (F) Effect of Oxidant on the Yield FDCA

In this experiment, different oxidants were respectively added to a mixture of 2.5 mL of the dehydration mixture, 2.5 mL of acetonitrile and 50 mg of Cu—Mn₂O₄, and then the oxidation reaction was conducted at 70° C. for 12 hours.

Next, the moles of 5-HMF and FDCA in the obtained oxidation mixture were analyzed by HPLC equipped with UV-Vis detector. The conversion rate of 5-HMF (%) and the yield of FDCA (%) in each group were calculated according to the above equations (3) and (4), respectively.

TABLE 6
Experimental results of different oxidants

| | | | Conversion | |
|---|---|---|---|---|
| | | | rate of | Yield of |
| | | Oxidant | 5-HMF | FDCA |
| Group | Oxidant | amount | (%) | (%) |
| F1 | Oxygen (O₂) | 0.5 mL | 23.53 | 7.10 |
| F2 | Hydrogen peroxide | 0.5 mL | 100 | 21.69 |
| | (H₂O₂) | | | |
| F3 | Potassium permanganate | 0.5 mg | 100 | 35.87 |
| | (KMnO₄) | | | |
| F4 | Sodium chloride | 0.5 mg | 100 | 8.82 |
| | (NaClO₃) | | | |
| F5 | Tert-butyl | 0.5 mL | 100 | 87.37 |
| | hydroperoxide(^{t}BuOOH) | | | |

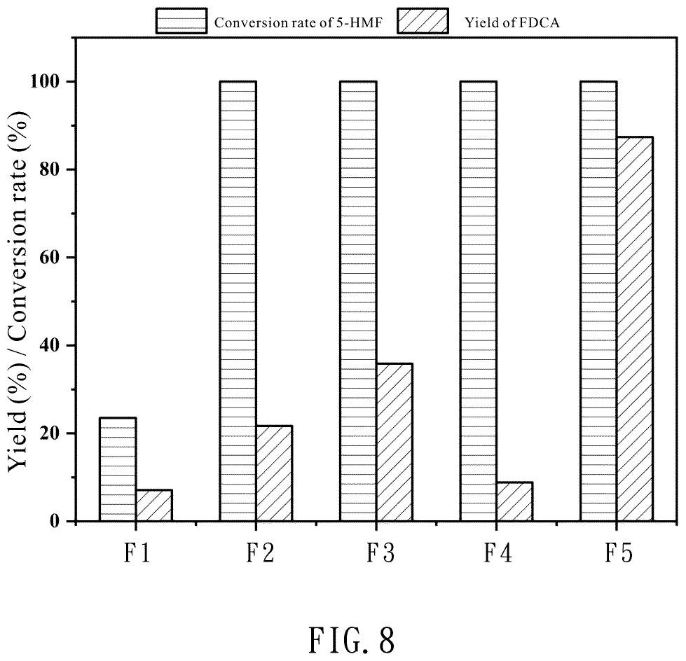

Referring to Table 6 and FIG. 8, among groups F1 to F5, group F5, which used tert-butyl hydroperoxide as the oxidant, exhibited the highest yield of FDCA.

### (G) Effect of Oxidant Amount on the Yield of FDCA

In this experiment, different amounts of tert-butyl hydroperoxide ranging from 0.3 to 0.9 mL were respectively added to a mixture of 2.5 mL of the dehydration mixture, 2.5 mL of acetonitrile and 50 mg of Cu—Mn₂O₄, and then the oxidation reaction was conducted at 70° C. for 12 hours.

Next, the moles of 5-HMF and FDCA in the obtained oxidation mixture were analyzed by HPLC equipped with UV-Vis detector. The conversion rate of 5-HMF (%) and the yield of FDCA (%) in each group were calculated according to the above equations (3) and (4), respectively.

TABLE 7
Experimental results of different amounts of oxidant

| | | Oxidant | Conversion | Yield of |
|---|---|---|---|---|
| | | amount | rate of 5-HMF | FDCA |
| Group | oxidant | (mL) | (%) | (%) |
| G1 | Tert-butyl | 0.3 | 88.23 | 52.16 |
| G2 | hydroperoxide | 0.5 | 100 | 78.64 |
| G3 | (^{t}BuOOH) | 0.7 | 100 | 83.21 |
| G4 | | 0.9 | 100 | 87.37 |

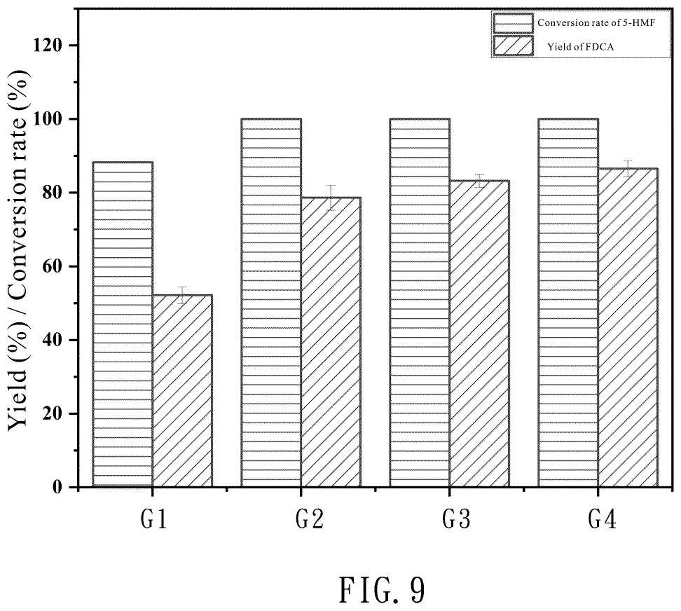

Referring to Table 7 and FIG. 9, in groups G1 to G4, the addition of 0.5 mL of tert-butyl hydroperoxide can result in a 100% conversion rate of 5-HMF, while group G4, with 0.9 mL of tert-butyl hydroperoxide, can increase the yield of FDCA to 87.37%.

### (H) Effect of Oxidation Catalyst Amount on the Yield of FDCA

In this experiment, different amounts of Cu—Mn₂O₄ ranging from 30 to 90 mg were respectively added as the oxidation catalyst to a mixture of 2.5 mL of the dehydration mixture, 2.5 mL of acetonitrile and 0.9 mL of tert-butyl hydroperoxide, and then the oxidation reaction was conducted at 70° C. for 12 hours.

Next, the moles of 5-HMF and FDCA in the obtained oxidation mixture were analyzed by HPLC equipped with UV-Vis detector. The conversion rate of 5-HMF (%) and the yield of FDCA (%) in each group were calculated according to the above equations (3) and (4), respectively.

TABLE 8
Experimental results of different amounts of oxidation catalyst

| | Oxidation | Conversion | Yield of |
|---|---|---|---|
| | catalyst amount | rate of 5-HMF | FDCA |
| Group | (mg) | (%) | (%) |
| H1 | 30 | 100 | 82.86 |
| H2 | 50 | 100 | 87.37 |
| H3 | 70 | 100 | 84.86 |
| H4 | 90 | 100 | 77.70 |

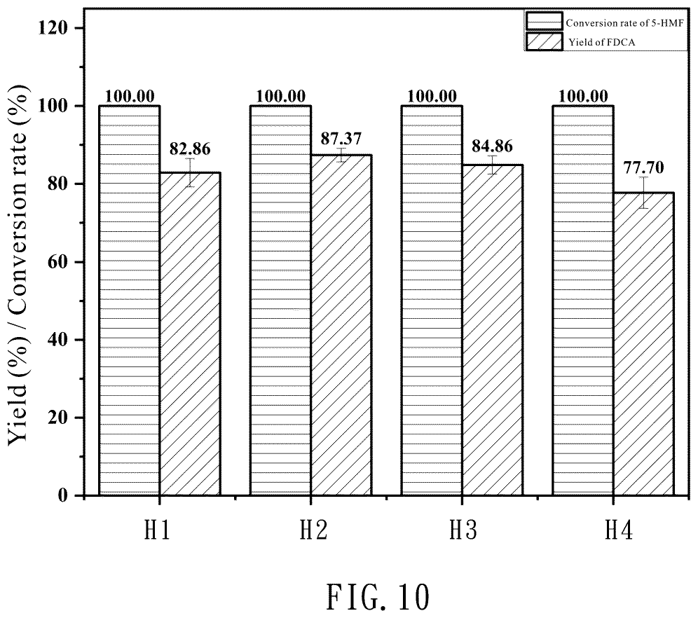

Referring to Table 8 and FIG. 10, in groups H1 to H4, the conversion rate of 5-HMF can reach 100% in all groups, while group H2, with 50 mg of Cu—Mn₂O₄, exhibited the highest yield of FDCA.

### (I) Effect of Reaction Temperature on the Yield of FDCA

In this experiment, a mixture of 2.5 mL of the dehydration mixture, 2.5 mL of acetonitrile, 50 mg of Cu—Mn₂O₄ and 0.9 mL of tert-butyl hydroperoxide was used to conduct the oxidation reaction at different temperatures ranging from 60 to 90° C. for 12 hours.

Next, the moles of 5-HMF and FDCA in the obtained oxidation mixture were analyzed by HPLC equipped with UV-Vis detector. The conversion rate of 5-HMF (%) and the yield of FDCA (%) in each group were calculated according to the above equations (3) and (4), respectively.

TABLE 9
Experimental results for different reaction temperatures

| | Reaction | Conversion | Yield of |
|---|---|---|---|
| | temperature | rate of 5-HMF | FDCA |
| Group | (° C.) | (%) | (%) |
| I1 | 60 | 99.10 | 63.11 |
| I2 | 70 | 100 | 87.37 |
| I3 | 80 | 100 | 80.17 |
| I4 | 90 | 100 | 78.97 |

Referring to Table 9 and FIG. 11, in groups I1 to I4, the conversion rate of 5-HMF can reach over 95% in all groups, while group 12, which conducted the oxidation reaction at 70° C., exhibited the highest yield of FDCA.

### (J) Effect of Reaction Time on the Yield of FDCA

In this experiment, a mixture of 2.5 mL of the dehydration mixture, 2.5 mL of acetonitrile, 50 mg of Cu—Mn₂O₄ and 0.9 mL of tert-butyl hydroperoxide was used to conduct the oxidation reaction at 70° C. for different reaction times ranging from 2 to 12 hours.

Next, the moles of 5-HMF and FDCA in the obtained oxidation mixture were analyzed by HPLC equipped with UV-Vis detector. The conversion rate of 5-HMF (%) and the yield of FDCA (%) in each group were calculated according to the above equations (3) and (4), respectively.

TABLE 10
Experimental results for different reaction times

| | | Conversion | Yield of |
|---|---|---|---|
| | Reaction time | rate of 5-HMF | FDCA |
| Group | (hr) | (%) | (%) |
| J1 | 2 | 77.40 | 16.13 |
| J2 | 4 | 94.95 | 39.75 |
| J3 | 6 | 99.02 | 71.36 |
| J4 | 8 | 100 | 76.97 |
| J5 | 10 | 100 | 84.16 |
| J6 | 12 | 100 | 87.37 |

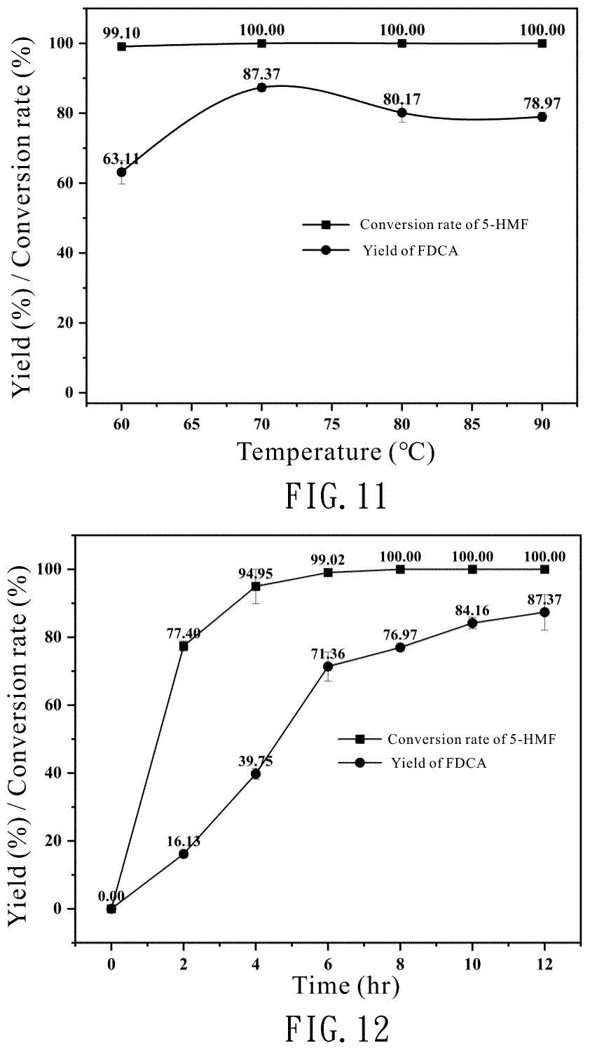

Referring to Table 10 and FIG. 12, among groups J1 to J6, group J6, which reacted for 12 hours, exhibited the highest yield of FDCA.

### (K) Effect of Different Biomass Materials

In this experiment, 0.2 g of different biomass materials were added to a mixture of 0.242 mL of [BMIM]HSO₄, 0.121 g of SiO₂/Al₂O₃, 7.5 mL of DMSO and 2.5 mL of deionized water, and then the dehydration reaction was conducted at 150° C. for 250 minutes. The concentration of 5-HMF in the obtained dehydration mixture was analyzed by HPLC equipped with UV-Vis detector. The yield of 5-HMF in each group was calculated according to the above equation (1).

Next, a mixture of 2.5 mL of the dehydration mixture, 2.5 mL of acetonitrile, 5011 mg of Cu—Mn₂O₄ and 0.9 mL of tert-butyl hydroperoxide was used to conduct the oxidation reaction at 70° C. for 12 hours. The moles of 5-HMF and FDCA in the obtained products were analyzed by HPLC equipped with UV-Vis detector. The conversion rate of 5-HMF (%) and the yield of FDCA (%) in each group were calculated according to the above equations (3) and (4), respectively.

TABLE 11
Experimental results of different biomass materials

| | | Dehydration | Oxidation reaction | | |
|---|---|---|---|---|---|
| | | reaction | Conversion | | Two-step |
| | | Yield of | rate of | Yield of | overall |
| | Biomass | 5-HMF | 5-HMF | FDCA | yield |
| Group | material | (%) | (%) | (%) | (%) |
| K1 | Jackfruit | 83.68 | 100 | 87.37 | 73.41 |
| | peel | | | | |
| K2 | Pineapple | 54.42 | 100 | 68.50 | 37.28 |
| | stem | | | | |
| K3 | Bagasse | 36.16 | 100 | 62.20 | 21.27 |
| K4 | Rice stalk | 12.82 | 100 | 69.38 | 8.98 |

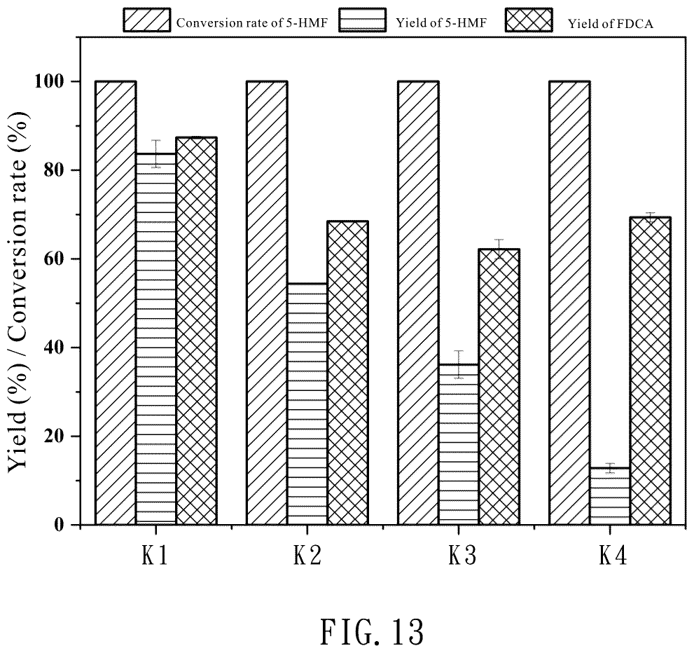

Referring to Table 11 and FIG. 13, among groups K1 to K4, group K1, which used jackfruit peel as biomass material, exhibited the highest yields of both 5-HMF and FDCA.

In view of the forgoing, the method for preparing 2,5-furandicarboxylic acid of the present invention allows the use of biomass material containing cellulose or lignocellulose as starting materials. Without the need for chemical pretreatment, high-value 2,5-furandicarboxylic acid (FDCA) can be formed through a two-step one-pot reaction. This method not only eliminates the need for the purification step of 5-hydroxymethylfurfural (5-HMF) and the use of noble metal catalysts, but also enables the synthesis of FDCA under mild conditions without strong bases or high pressure. Additionally, since the oxidation catalyst is a solid catalyst, it can be separated from the FDCA dissolved in the second solvent by simple filtration. The separated oxidation catalyst can be recycled and reused in catalyzing the oxidation reaction. Furthermore, the one-pot reaction system can simplify the complicated process for producing FDCA, thereby reducing its production cost.

Moreover, the method for preparing 2,5-furandicarboxylic acid of the present invention utilizes biomass material derived from agricultural waste to synthesize FDCA, which allows for the conversion of useless agricultural waste into high-value FDCA, thereby providing new economic value to the biomass material.

In addition, the method for preparing 2,5-furandicarboxylic acid of the present invention uses a dual solvent system of dimethyl sulfoxide (DMSO) and acetonitrile in a one-pot reaction, thereby enhancing the yield of 5-HMF in the dehydration reaction and the yield of FDCA in the oxidation reaction, respectively.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims. Furthermore, in a case that several of the above embodiments can be combined, the present invention includes the implementation of any combination.

What is claimed is:

1. A method for preparing 2,5-furandicarboxylic acid, comprising following steps:
mixing a biomass material, a dehydration catalyst and a first solvent to obtain a dehydration precursor, wherein the biomass material contains cellulose, and wherein the first solvent contains an ionic liquid, dimethyl sulfoxide and deionized water;
conducting a dehydration reaction on the dehydration precursor at 120-170° C. for 30-250 minutes to obtain a dehydration mixture containing 5-hydroxymethylfurfural;
mixing the dehydration mixture, an oxidation catalyst, an oxidant and a second solvent to obtain an oxidation precursor, wherein the oxidation catalyst is a solid copper-manganese nanomaterial; and
conducting an oxidation reaction on the oxidation precursor at 60-90° C. for 2-12 hours to obtain an oxidation mixture containing 2,5-furandicarboxylic acid.

2. The method for preparing 2,5-furandicarboxylic acid as claimed in claim 1, wherein the biomass material is jackfruit peel, pineapple stem, rice stalk or bagasse.

3. The method for preparing 2,5-furandicarboxylic acid claimed in claim 1, wherein the dehydration catalyst is a solid acid catalyst.

4. The method for preparing 2,5-furandicarboxylic acid as claimed in claim 3, wherein, after obtaining the dehydration mixture, the dehydration catalyst is separated from the dehydration mixture by filtration.

5. The method for preparing 2,5-furandicarboxylic acid as claimed in claim 1, wherein, by weight percentage, the dehydration precursor contains 0.13-3.68% of the biomass material, 0.78-4.50% of the dehydration catalyst and 92.10-99.08% of the first solvent.

6. The method for preparing 2,5-furandicarboxylic acid as claimed in claim 1, wherein, by volume percentage, the first solvent contains 0.8-4.6% of the ionic liquid, 24.4-74.1% of dimethyl sulfoxide and 24.4-74.1% of deionized water.

7. The method for preparing 2,5-furandicarboxylic acid as claimed in claim 1, wherein the ionic liquid is 1-allyl-3-methylimidazolium chloride ([AMIM]Cl), 1-butyl-3-methylimidazolium chloride ([BMIM]Cl), 1-butyl-3-methylimidazolium hydrogen sulfate ([BMIM]HSO₄) or 1-butyl-3-methylimidazolium tetrafluoroborate ([BMIM]BF₄).

8. The method for preparing 2,5-furandicarboxylic acid as claimed in claim 7, wherein the ionic liquid is 1-butyl-3-methylimidazolium hydrogen sulfate ([BMIM]HSO₄).

9. The method for preparing 2,5-furandicarboxylic acid as claimed in claim 1, wherein, after obtaining the oxidation mixture, the oxidation catalyst is separated from the oxidation mixture by filtration.

10. The method for preparing 2,5-furandicarboxylic acid as claimed in claim 1, wherein, by weight percentage, the oxidation precursor contains 25.66-69.51% of the dehydration mixture, 0.56-4.17% of the oxidation catalyst, 5.80-31.67% of the oxidant and 18.64-59.99% of the second solvent.

11. The method for preparing 2,5-furandicarboxylic acid as claimed in claim 1, wherein the oxidant is oxygen, hydrogen peroxide, potassium permanganate, sodium chloride or tert-butyl hydroperoxide.

12. The method for preparing 2,5-furandicarboxylic acid as claimed in claim 11, wherein the oxidant is tert-butyl hydroperoxide.

13. The method for preparing 2,5-furandicarboxylic acid as claimed in claim 1, wherein the second solvent is water, acetonitrile, ethyl acetate or tert-butanol.

14. The method for preparing 2,5-furandicarboxylic acid as claimed in claim 13, wherein the second solvent is acetonitrile.
